(19) United States
(12) Patent Application Publication
(10) Pub. No.: US 2025/0009209 A1
(43) Pub. Date: Jan. 9, 2025

(54) MEDICAL DEVICE ACTUATORS

(71) Applicant: Boston Scientific Scimed, Inc., Maple Grove, MN (US)

(72) Inventor: Sean POWELL, Holden, MA (US)

(73) Assignee: Boston Scientific Scimed, Inc., Maple Grove, MN (US)

(21) Appl. No.: 18/761,763

(22) Filed: Jul. 2, 2024

Related U.S. Application Data
(60) Provisional application No. 63/511,899, filed on Jul. 5, 2023.

(51) Int. Cl. A61B 1/00

(52) U.S. Cl. CPC A61B 1/00133

(57) ABSTRACT

An actuation mechanism for a medical device comprises: a rotatable member configured to rotate about a rotational axis and a spring, coupled directly or indirectly to the rotatable member. The spring is configured to move from a first configuration to a second configuration. In the first configuration, a longitudinal axis of the spring has a first angle with respect to a lever arm of the rotatable member. In the second configuration, a longitudinal axis of the spring has a second angle with respect to the lever arm of the rotatable member.

### CROSS-REFERENCE TO RELATED APPLICATIONS

This application claims the benefit of priority of U.S. Provisional Patent Application No. 63/511,899, filed on Jul. 5, 2023, the entirety of which is incorporated herein by reference.

### TECHNICAL FIELD

Various aspects of this disclosure relate generally to devices and methods for actuators of medical devices, and, in particular, to actuators having springs that help to decrease a perceived torque or force for a user as the actuator is operated.

### BACKGROUND

Medical devices, such as scopes (e.g., endoscopes, duodenoscopes, etc.), may include a handle and a sheath/shaft insertable into a body lumen of a subject. The sheath may terminate in a distal tip portion, which may include features such as elevators, optical elements (e.g., camera, lighting, etc.), air/water outlets, working channel openings, and/or accessory devices. Actuators in a handle of the scope may control actuatable elements of the shaft and/or distal tip. For example, buttons, knobs, levers, etc. may control elements of the shaft distal tip. Control elements (e.g., wires, cables, or shafts) may be utilized to transmit a force to a distal portion of the sheath. Such control elements may be coupled to control mechanisms in a handle, such as pulleys or levers. When a control mechanism is actuated by an actuator, a force may be transferred via a control element, thereby actuating elements of the shaft or distal tip. A need exists for improved actuation assemblies to transmit such forces from the handle of a medical device to distal portion of a sheath.

### SUMMARY

Each of the aspects disclosed herein may include one or more of the features described in connection with any of the other disclosed aspects.

Aspects of the disclosure relate to, among other things, systems, devices, and methods for actuators of medical devices.

In an aspect, an actuation mechanism for a medical device comprises: a rotatable member configured to rotate about a rotational axis and a spring, coupled directly or indirectly to the rotatable member. The spring is configured to move from a first configuration to a second configuration. In the first configuration, a longitudinal axis of the spring has a first angle with respect to a lever arm of the rotatable member. In the second configuration, a longitudinal axis of the spring has a second angle with respect to the lever arm of the rotatable member.

Any of the aspects disclosed herein may have any of the features below, alone or in any suitable combination. The mechanism further comprises a plunger coupled to the rotatable member at a connection. A portion of the plunger extends through a lumen of the spring. The actuation mechanism further comprises an anchor. A distal end of the spring is coupled to the anchor. The plunger extends through and is movable relative to the anchor. The plunger includes a protrusion. A proximal end of the spring engages with the protrusion. The first angle is smaller than the second angle. A first torque exerted by the spring on the rotatable member in the first configuration is smaller than a second torque exerted by the spring on the rotatable member in the second configuration. In a relaxed configuration of the actuation mechanism, a longitudinal axis of the spring is approximately coaxial with the lever arm of the rotatable member. The rotatable member includes a pulley. The actuation mechanism further includes a wire, cable, chain, or belt coupled to the pulley. The actuation mechanism further includes a linkage arm coupled to the rotatable member. The linkage arm is coupled to a control wire. The actuation mechanism further comprises a plunger coupled to the rotatable member at a connection. A portion of the plunger extends through a lumen of the spring. The linkage arm is coupled to the rotatable member at the connection. The connection is a first connection. The linkage arm is coupled to the rotatable member at a second connection. The second connection is at a different location than the first connection. A distal end of the plunger is coupled to a control wire. The spring is a coil spring, a leaf spring, or a hinge spring. The rotatable member is a first rotatable member. The lever arm is a first lever arm. The spring is a first spring. The actuation mechanism further comprises a second rotatable member configured to rotate about the rotational axis and a second spring directly or indirectly to the rotatable member. The second spring is configured to move from a first configuration to a second configuration. In the first configuration, a longitudinal axis of the second spring has a first angle with respect to a second lever arm of the second rotatable member. In the second configuration, a longitudinal axis of the second spring has a second angle with respect to the second lever arm of the second rotatable member. the rotatable member is coupled to a lever or a knob. A torque exerted by the spring on the rotatable member in the second configuration decreases an amount of force required for an operator to actuate the rotatable member.

In an example, an actuation mechanism for a medical device may comprise a rotatable member configured to rotate about a rotational axis, a plunger coupled to the rotatable member at a connection, and a spring. A portion of the plunger may extend through a lumen of the spring. The spring may be configured to move from a first configuration to a second configuration. In the first configuration, a longitudinal axis of the spring may have a first angle with respect to a line extending between the rotational axis and the connection. In the second configuration, a longitudinal axis of the spring may have a second angle with respect to a line extending between the rotational axis and the connection.

Any of the devices or mechanisms disclosed herein may have any of the following features, alone or in any combination or subcombination. The actuation mechanism may further comprise an anchor. A distal end of the spring may be coupled to the anchor. The plunger may extend through and be movable relative to the anchor. The plunger may include a protrusion. A proximal end of the spring may engage with the protrusion. The first angle may be smaller than the second angle. A first torque exerted by the spring on the rotatable member in the first configuration may be smaller than a second torque exerted by the spring on the rotatable member in the second configuration. In a relaxed configuration of the actuation mechanism, a longitudinal axis of the spring may be approximately coaxial with a line extending from the rotational axis to the connection. The rotatable member may include a pulley. The actuation mechanism may further include a wire, cable, chain, or belt coupled to the pulley. The actuation mechanism may further include a linkage arm coupled to the rotatable member. The linkage arm may be coupled to a control wire. The linkage arm may be coupled to the rotatable member at the connection. The connection may be a first connection. The linkage arm may be coupled to the rotatable member at a second connection. The second connection may be at a different location than the first connection. A distal end of the plunger may be coupled to a control wire. The plunger may extend distally from the rotatable member. The rotatable member may be a first rotatable member. The plunger may be a first plunger. The spring may be a first spring. The actuation mechanism may further comprise: a second rotatable member configured to rotate about the rotational axis; a second plunger coupled to the second rotatable member at a second connection; and a second spring. A portion of the second plunger extends through a lumen of the spring. The second spring may be configured to move from a first configuration to a second configuration. In the first configuration, a longitudinal axis of the second spring may have a first angle with respect to a line extending between the rotational axis and the second connection. In the second configuration, a longitudinal axis of the second spring may have a second angle with respect to a line extending between the rotational axis and the second connection. The rotatable member may be coupled to a lever or a knob. A torque exerted by the spring on the rotatable member in the second configuration may decrease an amount of force required for an operator to actuate the rotatable member.

In another example, an actuation mechanism for a medical device may comprise a rotatable member configured to rotate about a rotational axis and a spring configured to exert a force on the rotatable member. The spring may be configured to move from a first configuration to a second configuration. In the first configuration, the spring may exert a force along a first force vector, such that a first lever arm between the first force vector and the rotational axis has a first length. In the second configuration, the spring may exert a force along a second force vector, such that a second lever arm between the second force vector and the rotational axis has a second length. The first length may be smaller than the second length.

Any of the devices or mechanisms disclosed herein may have any of the following features, alone or in any combination or subcombination. The actuation mechanism may further comprise a plunger. The plunger may be coupled to the rotatable member. The plunger may extend through a lumen of the spring. A distal end of the plunger may extend through an anchor. A distal end of the spring may be coupled to the anchor.

In a further example, an actuation mechanism for a medical device may comprise: a rotatable member configured to rotate about a rotational axis, a plunger coupled to the rotatable member at a connection, and a spring. A portion of the plunger may extend through a lumen of the spring. The actuation mechanism may further comprise an anchor coupled to a distal end of the spring. As the rotatable member rotates about the rotational axis, the connection may follow an arcuate or circular path around the rotational axis.

Any of the devices or mechanisms disclosed herein may have any of the following features, alone or in any combination or subcombination. As the rotatable member rotates about the rotatable axis, a distance between a force vector of the spring and the rotational axis may change.

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “distal” refers to a direction away from an operator/toward a treatment site, and the term “proximal” refers to a direction toward an operator. One or more of the drawings may include arrows labeled “P” and “D,” referring to proximal and distal directions, respectively. The term “exemplary” is used in the sense of “example,” rather than “ideal.” The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1A shows a side perspective view of a medical device, according to some embodiments.

FIG. 1B shows a proximal end of the medical device of FIG. 1A, according to some embodiments.

FIG. 2 shows a cross-sectional side view of a handle of a medical device having an actuation mechanism, according to some embodiments.

FIGS. 3A-3C show partial cross-sectional views of the actuation mechanism of the handle of FIG. 2, according to some embodiments.

FIG. 4 depicts an alternative actuation mechanism.

FIGS. 5A-5B show side views of a lever assembly, according to some embodiments.

FIG. 6 shows a side view of a lever assembly, according to some embodiments.

FIG. 7 shows a cross-sectional side view of a handle of a medical device having an alternative actuation mechanism, according to some embodiments.

FIGS. 8A and 8B show cross-sectional side views of a handle of a medical device having a further alternative actuation mechanism, according to some embodiments.

FIGS. 9A and 9B show cross-sectional side views of a handle of a medical device having another alternative actuation mechanism, according to some embodiments.

FIGS. 10A and 10B show cross-sectional side views of a handle of a medical device having a further alternative actuation mechanism, according to some embodiments.

FIG. 11 shows a graph depicting aspects of the mechanisms of FIGS. 3A-10B, according to some embodiments.

### DETAILED DESCRIPTION

Actuators for medical devices, for example actuators within the handles of medical devices, such as scopes or accessory devices, may be utilized to actuate various elements disposed at the distal portions of the medical devices, such as steerable sections (e.g., articulation joints), elevators, cauterizing wires, end effectors, or elements configured to actuate secondary devices, such as bands or stents. In some embodiments, the actuator may include a knob, crank, or lever, which may be coupled to an element at the distal tip (e.g., via a control element, such as a wire, cable, or shaft). The actuator may be operated while a shaft of the medical device is within a body lumen of a subject. The body lumen may have a tortuous shape (i.e., have bends therein). Furthermore, the medical device (or an insertion device, e.g., a scope, through which the medical device is inserted) may be articulated so as to bend in one or more directions. A force necessary to actuate elements at the distal tip of a medical device generally increases corresponding to the angle of deflection (either passive or actively articulated). Furthermore, in some instances, the amount of force needed to actuate an element incrementally may increase as that element is actuated. For example, raising an elevator of a duodenoscope from 60 to 70 degrees may require more force than raising the element from 50 degrees to 60 degrees. Similarly, as a medical device is actively articulated using an actuator, an amount of force required to move the actuator may increase with increased articulation.

Physicians, and other users/operators, may conduct multiple medical procedures over the course of a day. As a result, the high ergonomic forces imparted to the user while actuating elements of the medical device may result in user fatigue, which may impair user endurance. Accordingly, it may be desirable to decrease an amount of force required to actuate elements of the medical device in order to reduce ergonomic forces experienced by an operator during a medical procedure, and thus help improve endurance. It may be particularly desirable to decrease an amount of force required to incrementally move an actuator as the actuator is moved further along its range of motion.

As described in further detail below, an actuation assembly may include a rotatable member, a control member (e.g., a wire, cable, or shaft) coupled to the rotatable member, a plunger rotatably coupled to the rotatable member, and a spring extending around the plunger. The spring may be anchored at a distal end thereof, thereby generating a reaction force of the spring against the anchor, which may provide a mechanical advantage along at least a portion of a rotation range of the rotatable member. The mechanical advantage may result in less force being required by a user to actuate the actuation assembly. This may, in turn, reduce a force necessary to change a configuration of an element at or near a distal tip of a medical device.

Although the term duodenoscope may be used herein, it will be appreciated that the disclosure encompasses actuation mechanisms for other devices, including, but not limited to, endoscopes, colonoscopes, ureteroscopes, bronchoscopes, laparoscopes, cytoscopes, hysteroscopes, sheaths, catheters, any other suitable delivery device, an accessory device for use with a delivery device (such as a scope), or another type of medical device including an actuator. Although side-facing devices may be referenced, the embodiments described herein may also be used with front-facing devices (e.g., devices where a viewing element faces longitudinally forward) or with devices that are a combination of side-facing and forward-facing. Although the lever assemblies described below are described as being used to articulate a distal portion of a medical device and/or raise/lower an element at the distal tip (e.g., an elevator), it will be appreciated that the lever assemblies may also be used to control other medical device components (e.g., end effectors, accessory devices, etc.).

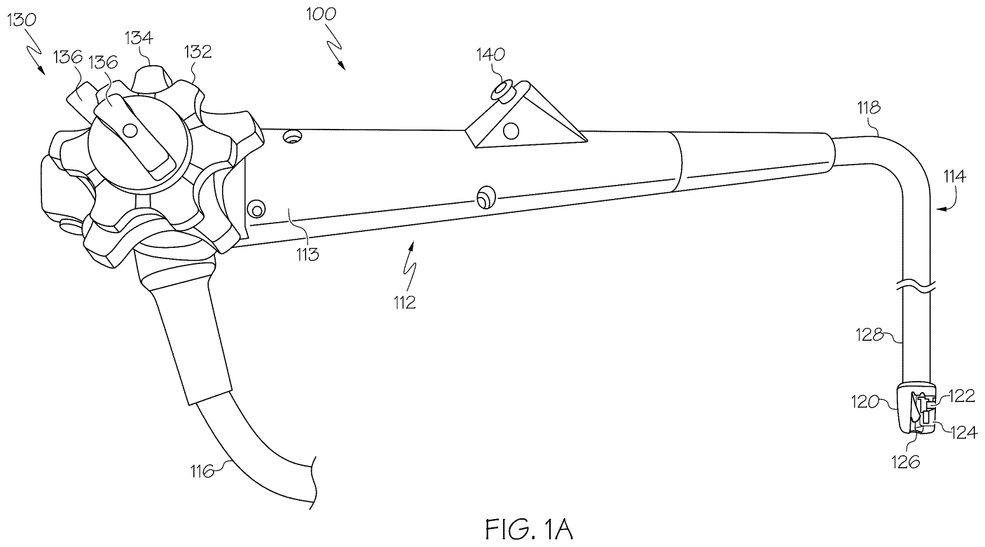

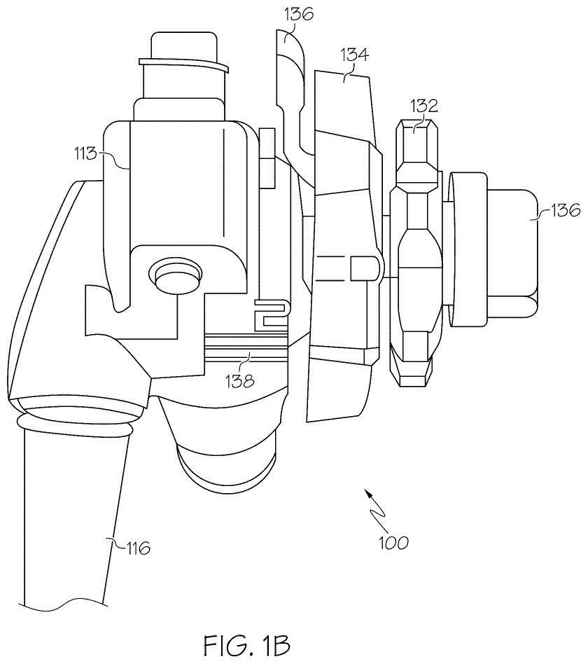

FIG. 1A depicts an exemplary medical device 100 (e.g., a duodenoscope) having a handle 112 and an insertion portion 114. FIG. 1B shows a proximal end of handle 112. Medical device 100 may also include an umbilicus 116 for purposes of connecting medical device 100 to sources of, for example, air, water, suction, power, etc., as well as to image processing and/or viewing equipment.

Insertion portion 114 may include a sheath or shaft 118 and a distal tip 120. Distal tip 120 may include an imaging device 122 (e.g., a camera) and a lighting source 124 (e.g., an LED or an optical fiber). Distal tip 120 may be side-facing. That is, imaging device 122 and lighting source 124 may face radially outward, perpendicularly, approximately perpendicularly, or otherwise transverse to a longitudinal axis of shaft 118 and distal tip 120. Alternatively, distal tip 120 may be forward-facing (imaging device 122 and/or lighting source 124 may face distally).

Distal tip 120 may also include an actuatable element, for example an elevator 126 for changing an orientation of a tool inserted in a working channel of medical device 100. Elevator 126 may alternatively be referred to as a swing stand, pivot stand, raising base, or any suitable other term. Elevator 126 may be pivotable via, e.g., an actuation wire or another control element that extends from handle 112, through shaft 118, to elevator 126.

A distal portion of shaft 118 that is connected to distal tip 120 may have a steerable section 128. Steerable section 128 may include, for example, an articulation joint. Shaft 118 and steerable section 128 may include a variety of structures which are known or may become known in the art. In some examples, one or more steering wires may be coupled to steerable section 128 so that, as the steering wires are tensioned/de-tensioned, steerable section 128 articulates in one or more directions.

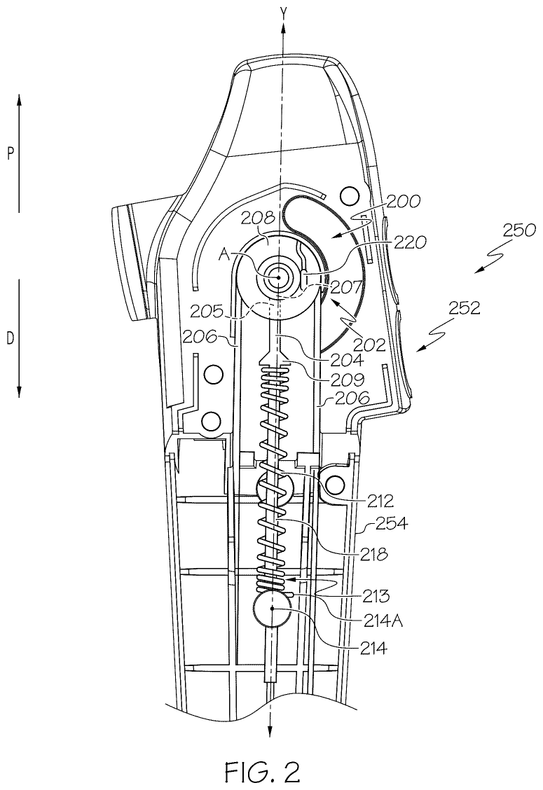

Handle 112 may have a housing 113 that encases/houses various elements of handle 112. Handle 112 may have one or more actuators/control mechanisms 130. Control mechanisms 130 may provide control over steerable section 128 or may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Handle 112 may further include one or more locking mechanisms 136 (e.g., knobs or levers) for preventing steering of steerable section 128 in at least one of an up, down, left, or right direction. Handle 112 may include an elevator control mechanism 138, such as a lever (FIG. 1B). Elevator control mechanism 138, may raise and/or lower elevator 126. For example, elevator control mechanism 138 may be coupled to a wire, which may be coupled to elevator 126. FIGS. 2-4C depict exemplary actuation mechanisms associated with one or more of elevator control mechanism 138 and/or knobs 132, 134. A port 140 may allow passage of a tool through port 140, into a working channel of the medical device 100, through shaft 118, to distal tip 120.

In use, an operator may insert at least a portion of shaft 118 into a body lumen of a subject. Distal tip 120 may be navigated to a procedure site in the body lumen. The operator may insert an accessory device, such as an instrument (not shown) into port 140, and pass the tool through shaft 118 via a working channel to distal tip 120. The tool may exit the working channel at distal tip 120. The user may use elevator control mechanism 138 to raise elevator 126 and angle the accessory device toward a desired location (e.g., a papilla of the pancreatico-biliary tract). The user may use the accessory device to perform a medical procedure.

Although FIGS. 1A-1B depict a side-viewing duodenoscope, it will be appreciated that, as discussed above, this disclosure is not so limited. The aspects discussed herein may alternatively be utilized with other types of medical devices. For example, the embodiments disclosed herein may be utilized with any type of scope. Alternatively, the embodiments disclosed herein may be utilized with other types of medical devices that involve actuating a control member (such as a wire, cable, rod, or other type of shaft) to control a portion of the medical device. Non-limiting examples of such medical devices include snares, staplers, graspers, catheters, tomes, stents, clips, balloons, baskets, forceps, knives, electrodes, or needles.

FIG. 2 depicts an exemplary actuation assembly/mechanism 200. Although FIG. 2 depicts an actuation mechanism 200 that may be used to articulate a steerable section, such as steerable section 128, it will be appreciated that the features discussed herein are broadly applicable to actuators of medical devices that may be rotated in order to control aspects of the medical device. Actuation mechanism 200 may be configured to decrease a torque experienced by an operator in operating actuation mechanism 200. As shown in FIG. 2, actuation mechanism may be at least partially disposed within a housing 254 of a handle 252 of a medical device 250. Handle 252 may have any of the features of handle 112 of medical device 100. Medical device 250 may further include an insertion portion (not shown) extending distally from handle 252 and having any of the features of insertion portion 114, discussed above.

In some embodiments, for example as shown in FIG. 2, actuation mechanism 200 may include a pulley system 202. Pulley system 202 may include a rotatable member 208. Rotatable member 208 may be formed from any suitable material, such as, for example, rigid materials, such as plastic or other polymers, composites, or metal. Rotatable member 208, as depicted in FIG. 2, may be a pulley. Alternatively, rotatable member 208 may be a spool, crank, lever, gear (e.g., pinion gear and/or rack), of other type of rotatable element. Rotatable member 208 may be coupled to one or more elements that may be contacted by a user in order to rotate rotatable member 208 (e.g., knobs, levers, cranks, etc., such as control knobs 132, 134 of FIGS. 1A-1B) or elevator control mechanism 138. In some embodiments, as shown in FIG. 2, at least one wire or cable 206 (or chain or belt) may be coupled to rotatable member 208. In some aspects, and as shown in FIG. 2, two cables 206 may be coupled to rotatable member 208. For example, as shown in FIG. 2, cable(s) 206 may wrap around rotatable member 208 (e.g., a proximal portion of rotatable member 208), and may be attached to rotatable member 208 via an attachment point 220. In some embodiments, attachment point 220 may include a ferule, or any other suitable means for attaching cables 206 to rotatable member 208. Cables 206 may be further configured to be coupled to an actuatable element disposed in a shaft/at a distal tip of medical device 250. For example, cables 206 may be coupled to an articulation joint of, for example, a steerable section, such as steerable section 128. When cables 206 are moved proximally or distally via rotation of rotatable member 208, a distal portion of the medical device (e.g., steerable section 128 of medical device 100) may deflect in one or more directions.

Rotatable member 208 also may be coupled to a plunger 204. A first end 205 (e.g., a proximal end) of plunger 204 may be rotatably coupled to rotatable member 208 via a connection 207. Connection 207 may be radially offset from a rotational axis A of rotatable member 208. As shown in FIG. 2, connection 207 may be positioned between rotational axis A and an outer circumference of rotatable member 208. Alternatively, connection 207 may be on or near an outer circumference or edge of rotatable member 208.

A distal portion of plunger 204 may extend through or around an anchor 214. In some examples, plunger 204 may extend through anchor 214. In alternatives, plunger 204 may surround anchor 214. As discussed below, with respect to anchor 214′, anchor 214 may allow plunger 204 to move proximally/distally and constrain an amount of lateral movement of plunger 204. Anchor 214 may be disposed on a longitudinal axis of handle 252 and/or on a line extending through rotational axis A and connection 207.

In some embodiments, actuation mechanism 200 may additionally include a spring 212. In some examples, spring 212 may be a coiled spring, such as a helical compression spring. Windings of spring 212 may define a lumen through which a rod portion 218 of plunger 204 may extend. In other words, spring 212 may surround (i.e., extend around) rod portion 218. Thus, spring 212 may be indirectly coupled to rotatable member 208 via plunger 204. In some examples, a central longitudinal axis of spring 212 may be coaxial with a central longitudinal axis of plunger 204. A distal portion 213 of spring 212 may be fixed to anchor 214 or held in contact with anchor 214 by pressure. In some embodiments, anchor 214 may include a feature 214A (e.g., a protrusion or extension from a remainder of anchor 214), which may be configured to be directly coupled to a distalmost end of spring 212 or another part of distal portion 213 of spring 212. Because spring 212 is coupled to anchor 214, anchor 214 may provide a surface against which spring 212 may generate a reaction force. In some examples, plunger 204 may be omitted, and spring 212 may be anchored to other elements of actuation mechanism 200.

As shown in FIG. 2, a portion of plunger 204 (e.g., a proximal portion of plunger 204) may include a protrusion 209. A proximal end of spring 212 may engage (e.g., abut) protrusion 209 and may exert forces (e.g., restoring forces) on protrusion 209. Contact pressure between the proximal end of spring 212 may retain the proximal end of spring 212 against protrusion 209. Optionally, the proximal end of spring 212 may be fixedly coupled to protrusion 209. Spring 212 may be compressed relative to its neutral length, so that spring 212 exerts a force on protrusion 209 (and anchor 214). This force may be transmitted to rotatable member 208 via connection 207. As shown in FIG. 2, spring 212 may be indirectly coupled to rotatable member 208, via plunger 204. In alternatives, spring 212 may be directly coupled to rotatable member 208.

Still referring to FIG. 2, at least portions of spring 212 may be moveably aligned with plunger 204 along a longitudinal axis of plunger 204. In other words, intermediate windings of spring 212 (between proximal and distal ends of spring 212) may be movable relative to plunger 204, such that spring 212 may be able expand/compress along plunger 204 as plunger 204 moves with rotatable member 208. In a neutral position of actuation mechanism 200, a longitudinal axis of plunger 204 and/or spring 212 may be approximately parallel to or coaxial with a longitudinal axis Y of housing 254/medical device 250. As shown in FIG. 2, a longitudinal axis of plunger 204 and/or spring 212 may be approximately vertical in the neutral configuration. In the configuration shown in FIG. 2, spring 212 may exert a restoring force in a proximal direction on protrusion 209, along longitudinal axis Y. A central longitudinal axis of plunger 204 and/or spring 212 may extend through rotational axis A of rotatable member 208 in the neutral configuration (e.g., a configuration in which rotatable member 208 is not actuated). In other words, the central longitudinal axis of plunger 204 and/or spring 212 may be coaxial with a line extending between rotation axis A and connection 207. Thus, a vector of a restoring force from spring 212 may pass through rotational axis A of rotatable member 208 when in a neutral position, such as when actuation mechanism 200 is not engaged by a user. Accordingly, in a configuration where a longitudinal axis of plunger 204 and/or spring 212 extends through rotational axis A (i.e., when a longitudinal axis of plunger 204 and/or spring 212 is coaxial with a line extending between rotational axis A and connection 207), little to no torque is felt by a user via, e.g., control knobs 132, 134, as a result of spring 212 because the longitudinal axis of spring 212 extends along a radius of rotatable member 208 or approximately along a radius of rotatable member 208.

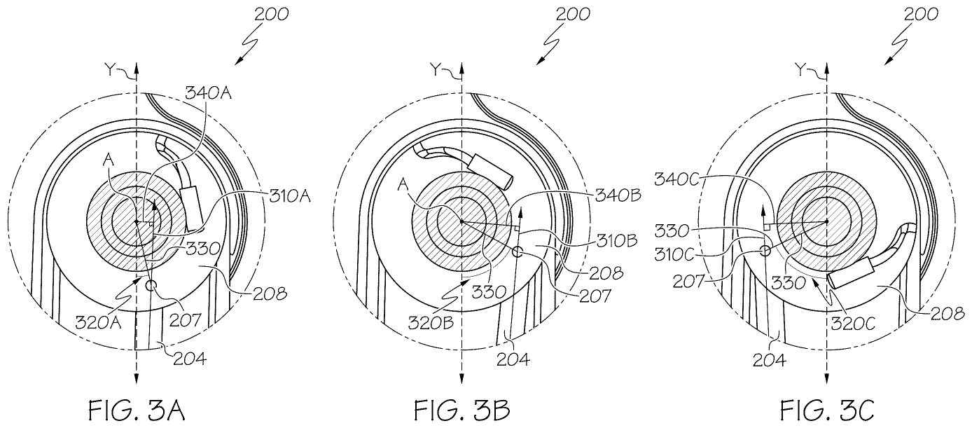

As discussed in further detail with respect to FIGS. 3A-3C, when a user engages actuation mechanism 200, for example by rotating a control knob (not shown), such as control knobs 132/134 shown in FIGS. 1A and 1B, the rotation of the control knob may cause rotation of rotatable member 208, which may result in cables 206 being shortened or lengthened. As discussed above, as the user engages actuation mechanism 200 to rotate rotatable member 208 by larger amounts, conventional devices may require a larger amount of force to be exerted by the user in order to continue to rotate rotatable member 208, which may cause fatigue in the user's hand after prolonged use. In actuation mechanism 200, however, rotation of rotatable member 208 may also cause an angle of spring 212 to change, as spring 212 may be configured to move along with plunger 204 as rotatable member 208 is rotated. For example, connection 207 may move along/follow a circular/circumferential or arcuate path in accordance with a distance between connection 207 and rotational axis A. Thus, first end 205 of plunger 204 may similarly follow a circular or arcuate path. When rotatable member 208 is rotated in a first direction, first end 205 of plunger 204 may travel around the circular or arcuate path in the first direction. When rotatable member 208 is rotated in a second direction, opposite to the first direction, first end 205 of plunger 204 may travel around the circular or arcuate path in the second direction.

Thus, an angle of plunger 204 and spring 212 may change as rotatable member 208 is rotated. In some configurations, central longitudinal axes of plunger 204 and/or spring 212 may be transverse (i.e., not parallel to or coaxial with) to a radius of rotatable member 208 extending between rotational axis A and connection 207 (i.e., a line extending between rotational axis A and connection 207). In other words, in the coordinate system of FIG. 2, plunger 204 and/or spring 212 may have a central longitudinal axis that is not vertical during portions of a rotational path of rotatable member 208 and is not otherwise aligned with the radius of rotatable member 208 extending through connection 207. Accordingly, a spring force vector of spring 212 may likewise be transverse to the radius of rotatable member 208 extending through connection 207. In other words, the spring force vector and/or longitudinal axis of spring 212 may not pass through rotational axis A of rotatable member 208. When plunger a longitudinal axis of spring 212 has a greater angle with respect to the radius extending through connection 207 (i.e., a greater angle with respect to a vertical direction in FIG. 2), an amount of torque generated by spring 212 may increase, as discussed below. The torque generated by spring 212 may help to reduce a force necessary to rotate rotatable member 208, for example, the force applied by the user to rotate rotatable member 208.

This concept is depicted, for example, in FIGS. 3A-3C, which show various angles of rotation of rotatable member 208 and corresponding force vectors generated by spring 212. FIGS. 3A-3C show a cross-section of a portion of actuation mechanism 200, through a center of rotatable member 208. For ease of illustration, elements such as spring 212 and housing 254 are omitted from FIGS. 3A-3C. It will be appreciated that actuation mechanism 200 may be incorporated into handle 252, as shown in FIG. 2.

FIG. 3A shows actuation mechanism 200 in a first configuration, which is a slightly off-neutral position. As described herein, for ease of discussion, a neutral position is one in which a longitudinal axis of plunger 204 extends through the rotational axis of rotatable member 208 (along a radius extending through connection 207). In such a configuration, connection 207 may be disposed at a 6 o'clock position of FIG. 3A (i.e., at a lowermost position). However, it will be appreciated that a neutral position of actuation mechanism 200 may actually be offset from such a position, either in a clockwise or counterclockwise direction.

As shown in FIG. 3A, rotatable member 208 has been rotated in a first direction (e.g., counterclockwise) by a small angle 320A relative to a longitudinal axis Y of actuation mechanism 200. Plunger 204 may similarly be deflected from the longitudinal axis Y of actuation mechanism 200 by a small angle that depends upon a length of plunger 204. Spring 212 (not shown in FIG. 3A) may be deflected by the same angle as plunger 204. Thus, a force vector 310A of spring 212 (not shown to scale in FIG. 3A, as force vector 310A is intended to show a direction only of force vector 310A and not a magnitude of force vector 310A) may extend along plunger 204, through connection 207. Force vector 310A may act on a rotatable member arm 330 of rotatable member 208 that extends from axis of rotation A of rotatable member 208 to connection 207. Because force vector 310A is deflected only slightly from longitudinal axis Y, a lever arm length 340A (i.e., a distance between force vector 310A and rotational axis A) may be relatively small. A magnitude of a torque exerted on rotatable member arm 330 by force vector 310A may be equal to a magnitude of force vector 310A multiplied by lever arm length 340A. The magnitude of the torque exerted on rotatable member arm 330 by force vector 310A may be relatively small compared to the torques discussed below for FIGS. 3B and 3C.

In FIG. 3B, rotatable member 208 of actuation mechanism 200 has been rotated in the first direction by an angle 320B relative to longitudinal axis Y. Angle 320B may be larger than angle 320A. Plunger 204/spring 212 may be deflected from the longitudinal axis of handle 252 by a larger angle than in FIG. 3B, such that a force vector 310B (again not drawn to scale and merely depicting a direction of the force of spring 212, rather than a magnitude of the force) is deflected further from longitudinal axis Y than force vector 310A of FIG. 3A. Because a distance from rotational axis A of rotatable member 208 to connection 207 does not change, a length of rotatable member arm 330 remains constant. However, a lever arm length 340B (a distance from force vector 310B to rotational axis A) may be relatively larger than lever arm length 340A. A magnitude of a torque exerted by force vector 310B may be equal to a magnitude of force vector 310B multiplied by lever arm length 340B. A magnitude of a torque exerted by force vector 310B may thus be larger than a torque exerted by force vector 310A because lever arm length 340B is larger than lever arm length 340A.

Spring 212 may have properties (e.g., a length and/or spring constant) such that a torque exerted spring 212 may be at a maximum prior to rotatable member arm 330 being perpendicular to an axis of plunger 204 (and a force vector of spring 212). With continued movement of rotatable member 208, a torque resulting from spring 212 may decrease. In some examples, rotatable member 208 may be limited to rotating less than or equal to approximately 90 degrees or less than or equal to approximately 120 degrees. In some examples, actuation mechanism 200 or a handle (such as handle 112) may provide a warning to an operator (e.g., via a visual indicator) that the operator is reaching an end of travel of rotatable member 208 without providing a hard stop.

FIG. 3C shows a third configuration, in which rotatable member 208 has been rotated by an angle 320C in a second direction, opposite of the first direction of FIGS. 3A and 3B. In some examples, angle 320C may be similar in size to angle 320B but in an opposite direction. Because a distance from the rotational axis of rotatable member 208 to connection 207 does not change, a length of rotatable member arm 330 remains constant. Spring 212 may exert a force vector 310C on rotatable member 208 (as above, force vector 310C is not drawn to scale and merely depicts a direction of the force exerted for illustration). A lever arm length 340C (a distance between force vector 310C and rotational axis A) may be similar to lever arm length 340B. A magnitude of a torque exerted by force vector 310C may be similar to a magnitude of a torque exerted by force vector 310B. Alternatively, depending on an angle between force vector 310C and rotatable member arm 330, a magnitude of a torque exerted by force vector 310C may be larger or smaller than a magnitude of a torque exerted by force vector 310B (and/or force vector 310A).

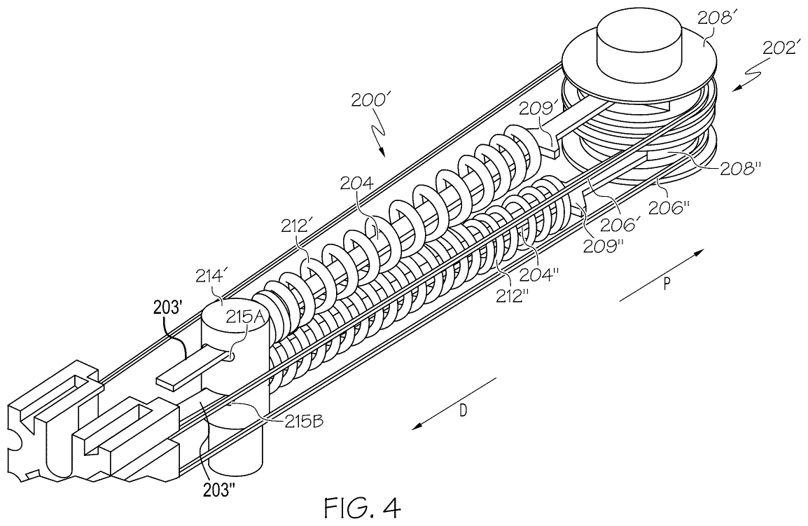

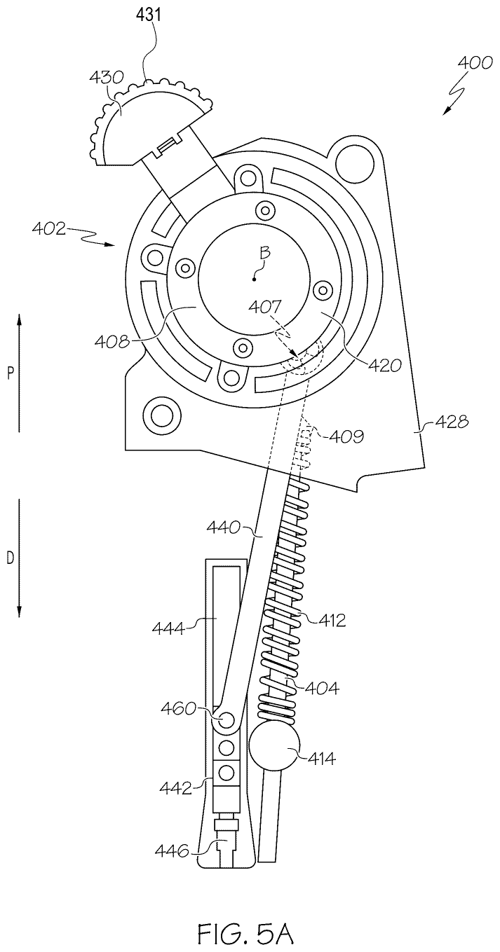

FIG. 4 depicts an alternative actuation mechanism 200′, which may be used with any of the handles described herein (e.g., handles 112, 252). Where feasible, reference numbers of FIG. 4 add a “prime” to reference numbers of FIG. 2 to denote corollary structures. As shown in FIG. 4, a pulley system 202′ may have two rotatable members 208′ and 208″. Rotatable members 208′, 208″ may be pulleys in some examples. Rotatable members 208′, 208″ may rotate about the same rotational axis. In alternatives, rotatable members 208′, 208″ may rotate about different rotational axes. Each rotatable member 208′, 208″ may have one or more wires or cables 206′, 206″, respectively, wrapped therearound or otherwise coupled thereto. One of rotatable members 208′, 208″ may be configured to selectively apply tension to the respective cable(s) 206′, 206″ in order to move a distal portion (e.g., shaft) of medical device 250′ in a first plane (e.g., a left/right plane). The other of rotatable members 208′, 208″ may be configured to selectively apply tension to the respective cable(s) 206′, 206″ in order to move a distal portion (e.g., shaft) of medical device 250′ in a second plane (e.g., an up/down plane). Each of two knobs (e.g., knobs 132, 134) or other actuators may be coupled to one of rotatable members 208′, 208″ in order to actuate rotatable members 208′, 208″. Alternative actuation mechanisms also may be utilized.

As shown in FIG. 4, each of rotatable members 208′, 208″ may have a plunger 204′, 204″, respectively, coupled thereto. Plungers 204′, 204″ may be coupled to rotatable members 208′, 208″ by any of the mechanisms discussed above for plunger 204 and rotatable member 208. Each of plungers 204′, 204″ may pass through an anchor 214′, having any of the properties of anchor 214, discussed above. As shown in FIG. 4, anchor 214′ may have two openings 215A, 215B for receiving plungers 204′, 204″, respectively, therethrough. In some examples, as shown in FIG. 4, plungers 204, 204″ may have a rectangular cross section. Plungers 204′, 204″ may be movable with respect to openings 215A, 215B, to accommodate movement of plungers 204′ and/or 204″ as rotatable members 208′ and/or 208″ rotate. Openings 215A, 215B may be sized and shaped so as to accommodate plungers 204′, 204″ and allow for a changing angle of plungers 204′, 204″ with respect to a longitudinal axis of actuation mechanism 200′. For example, openings 215A, 215B may be wider (e.g., slightly wider) in a lateral direction that plungers 204′, 204″ to allow for changing angles of plungers 204′, 204″ Distal ends 203′, 203″ of plungers 204′, 204″ respectively may extend distally of anchor 214′.

Actuation mechanism 200′ also may include springs 212′, 212″ associated with rotatable members 208′, 208″, respectively. As shown in FIG. 4, and as described above with respect to FIG. 2, plungers 204′, 204″ may extend through a lumen of a respective spring 212′, 212″. As discussed above, proximal ends of springs 212′, 212″ may exert a force on protrusions 209′, 209″ of respective plungers 204′, 204″. Protrusions 209′, 209″ may have any of the properties of protrusion 209. As shown in FIG. 4, protrusions 209′, 209″ may extend laterally outward from plungers 204′, 204″. Plunger 204′, 204″ and the respective protrusion 209′, 209″ may form cross shape, as depicted in FIG. 4.

Springs 212′, 212″ may function in the same manner as spring 212 in order to exert a torque on a respective rotatable member 208′, 208″ as rotatable member 208,′ 208″ is rotated (e.g., via a knob or lever, not shown in FIG. 4). As discussed above, a torque exerted by springs 212′, 212″ may be larger as rotatable members 208,′ 208″ are rotated by greater amounts relative to a longitudinal axis of actuation mechanism 200′.

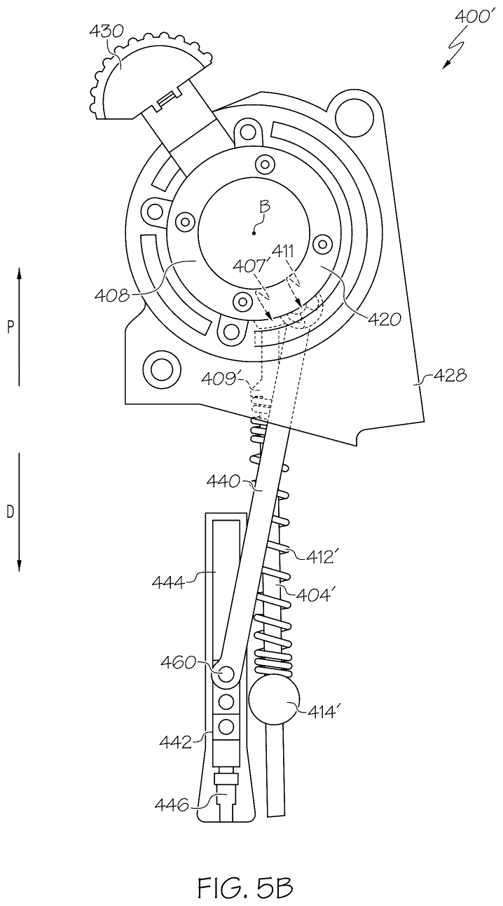

The exemplary actuation mechanisms 200, 200′ are described with rotatable members 208, 208′, 208″ being pulleys used to actuate steering wires/cables 206. However, such mechanisms are merely exemplary. The concepts of actuation mechanisms 200, 200′ may be applied to a wide variety of rotating actuation assemblies. For example, FIG. 5A shows an actuation mechanism 400 having a lever assembly 402 used to actuate an element at a distal end of a medical device. In some examples, lever assembly 402 may be used to raise/lower an elevator, such as elevator 126. Where feasible, reference numbers used in FIG. 5A add 200 to the reference numbers used in FIG. 2 to denote similar elements.

In some embodiments, lever assembly 402 may include a rotatable member 408 coupled to a linkage rod 440 and a plunger 404 (discussed in further detail below). Rotatable member 408 may also be coupled to a control lever 430, which may extend radially outward from rotatable member 408. Control lever 430 may have any of the properties of elevator control mechanism 138 (FIG. 1B). In some embodiments, rotatable member 408 may include an annular ring rotatable about a central axis B. Annular ring 420 may be disposed within a handle of a medical device, such as handle 112 (FIGS. 1A-1B) of medical device 100. Although lever assembly 402 is described as being an aspect of medical device 100, below, it will be appreciated that lever assembly 402 may alternatively be used with other medical devices.

Control lever 430 may extend outwardly through housing 113 of handle 112 via, e.g., a slit or opening in housing 113. Edges of the slit may define a range through which control lever 430 may be moved. Alternatively, other structures internally to or externally of housing 113 of handle 112 (e.g., a cover 428, discussed in more detail below) may constrain a range by which control lever 430 may be moved. Control lever 430 may have a range extending from an initial position, at one end of a range of control lever 430, to a final position, at the other end of a range of control lever 430. In some embodiments, control lever 430 may include ridges or a grip surface 431 for providing a contact surface for a user's finger, which may help to provide traction to a user. Annular ring 420 may be rotatable relative to housing 113 of handle 112, such that movement of control lever 430 causes rotation of annular ring 420 relative to housing 113 of handle 112. Annular ring 420 and control lever 430 may be a single, unitary structure, or annular ring 420 and control lever 430 may be separate elements attached to one another.

Components of rotatable member 408 may be formed of any suitable material. For example, components of rotatable member 408 may be formed of rigid materials, such as plastic or other polymers, composites, or metal. Control lever 430, and annular ring 420 may be formed from the same materials or from different materials.

As shown in FIG. 5A, cover 428 may cover at least a portion of annular ring 420. Cover 428 may provide for separation between annular ring 420 and other components of handle 112 and/or may be used to retain annular ring 420 within a desired plane. Annular ring 420 may be rotatable relative to cover 428. Cover 428 may be stationary relative to housing 113 of handle 112.

In some embodiments, proximal ends of each of linkage rod 440 and plunger 404 may be coupled to rotatable member 408 (e.g., to annular ring 420) at a connection 407. In some embodiments, a pin may secure linkage rod 440 and/or plunger 404 to rotatable member 408, such that linkage rod 440 and/or plunger 404 is/are rotatable and/or pivotable relative to rotatable member 408. In some examples, a same pin/other structure may be used to couple both of linkage rod 440 and plunger 404 to rotatable member 408. Alternatively, separate pins/other structures may be used to couple each of linkage rod 440 and plunger 404 to rotatable member 408. Linkage rod 440 and plunger 404 may each extend distally from rotatable member 408 within an interior of handle 112. As shown in FIG. 5A, linkage rod 440 may be straight; however, linkage rod 440 may have any suitable shape. Similarly, plunger 404 may also be straight or may have alternative shapes. Linkage rod 440 and plunger 404 may each be formed of any suitable material. For example, linkage rod 440 and plunger 404 may each be formed of rigid materials, such as plastic or other polymers, composites, or metal.

In some embodiments, a distal end of linkage rod 440 may be coupled to a slider 442. A pin or other structure may rotatably couple the distal end of linkage rod 440 to slider 442, for example, at connection point 460. Movement of slider 442 may be constrained by a channel 444. Channel 444 may extend approximately parallel to the longitudinal axis of handle 112. Channel 444 may help to inhibit slider 442 from moving in directions perpendicular to the longitudinal axis and may constrain slider 442 to longitudinal movement. Slider 442, in turn, may be coupled to a control member 446 (e.g., a wire). A distal end (not shown) of control member 446 may be coupled to, e.g., elevator 126. Thus, movement of control lever 430 may serve to raise/lower elevator 126. The elements described above are merely exemplary, and any actuation mechanism known in the art may be utilized to control elevator 126. In alternatives, control member 446 may be coupled to an alternative structure (e.g., end effector, articulation joint, cauterizing wire, or actuator for a secondary device, such as a band or a stent).

Plunger 404 may have any of the properties of plungers 204, 204′, or 204″. A spring 412, for example a coil spring, may surround plunger 404 in a similar manner to how spring 212 surrounds plunger 204 of pulley system 202, described above. Thus, spring 412 may be indirectly coupled to rotatable member 208 via plunger 404. Plunger 404 may extend through or around an anchor 414, having any of the properties of anchors 214, 214′, above. As discussed above, a distal end of spring 412 may be fixedly coupled to anchor 414. Plunger 404 may include a protrusion 409, similar to protrusions 209, 209′, 209″, discussed above. Spring 412 may engage with protrusion 409 in order to exert a force on plunger 404 and, thus, rotatable member 408. As discussed above, spring 412 may be compressed to less than its natural length, such that spring 412 exerts a force along a direction of plunger 404. For example, as shown in FIG. 5A, spring 412 may exert a force that is at least partially in a proximal direction on protrusion 409.

As control lever 430 is rotated, thereby rotating rotatable member 408 (including annular ring 420), proximal ends of plunger 404 and linkage rod 440 may move therewith. As a proximal end of plunger 404 moves circumferentially about rotatable member 408, an angle of plunger 404 and spring 412 may change, as discussed above for plunger 204 and spring 212, with respect to FIGS. 2-3C. Thus, a direction of a force vector exerted by spring 212 may change, along with a distance between the force vector and a rotational axis B of rotation and, thus, a torque exerted by spring 212. In some examples, lever 430 may be rotatable only in one direction (e.g., a direction of FIGS. 3A-3B or a direction of FIG. 3C). In alternatives, lever 430 may be rotatable in both directions (a first direction of FIGS. 3A-3B and a second direction of FIG. 3C). Spring 412 may thus provide torque to assist a user with rotating control lever 430. Thus, actuation mechanism 400 may reduce an effort required by an operator as control lever 430 is rotated by further amounts.

FIG. 5B shows an alternative actuation mechanism 400′. Actuation mechanism may have any of the properties of actuation mechanism 400, except as specified herein. Where feasible, reference numbers of FIG. 5B use the same reference number to identify the same parts and add a “prime” to reference numbers to identify similar elements. As depicted in FIG. 5A, linkage rod 440 and plunger 404 are coupled to rotatable member 408 at a same location of rotatable member 408 via connection 407. In contrast, actuation mechanism 400′ may include linkage rod 440 and a plunger 404′ coupled to rotatable member 408 at different locations on rotatable member 408. For example, as shown in FIG. 5B, plunger 404′ may be coupled to rotatable member 408 at a connection 407′. Linkage rod 440 may be coupled to rotatable member 408 at a connection 411. Connection 411 may be at a different location than connection 407′. For example, connection 407′ may be offset in a counterclockwise direction from connection 411. Alternatively, connection 407′ may be offset in a clockwise direction from connection 411.

Similarly to actuation mechanism 400, plunger 404′ may include a spring 412′ extending thereabout. Thus, spring 412′ may be indirectly coupled to rotatable member 408 via plunger 404′. A distal end of spring 412′ may be fixed relative to an anchor 414′, having any of the properties of anchors 214, 214′, or 414. As discussed above with respect to springs 212, 212′, 212″, 412, spring 412′ may be compressed with respect to its relaxed length, so that spring 412′ exerts a force on a protrusion 409′ (having any of the properties of protrusions 209, 209′, 209″, 409).

Connection 407′ and connection 411 may be arranged so that an axis of plunger 404′ extends through (or nearly extends through) rotational axis B in an unactuated state of lever 430 (e.g., in a configuration in which elevator 126 is fully lowered). In such a configuration, spring 212′ may not exert a torque on rotatable member 408 via a protrusion 409′ of plunger 404. Such a configuration may be shown in FIG. 5B. Relative positions of connection 407′ and connection 411 may be selected for a particular range of actuation mechanism 400′.

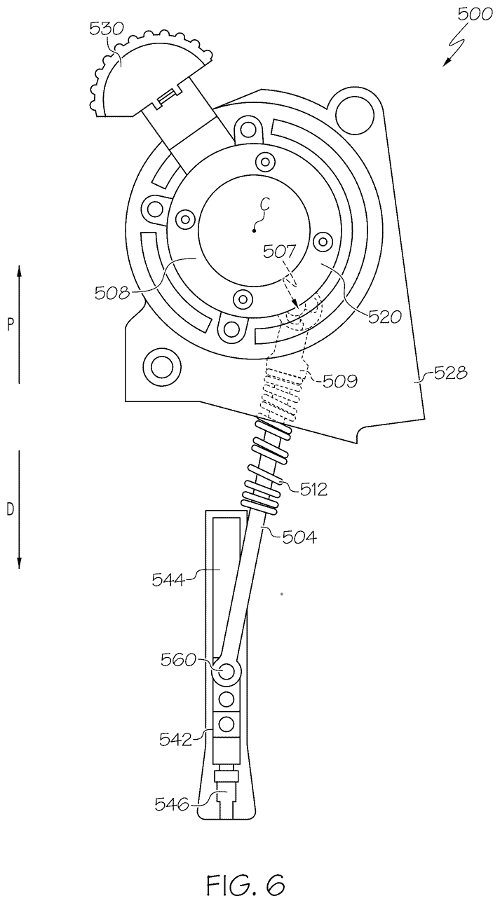

In some embodiments, for example as shown in FIG. 6, an actuation mechanism 500 may include a plunger 504 that includes the functionality of linkage rod 440. Where feasible, reference numbers of FIG. 6 add 100 to reference numbers of FIG. 5A. Like actuation mechanisms 400 and 400′, described above, actuation mechanism 500 may include a rotatable member 508 (having any of the properties of rotatable member 408) coupled to plunger 504 at a connection 507. Rotatable member 508 may also be coupled to a control lever 530, which may extend radially outward from rotatable member 508. Control lever 530 may include all of the features of control lever 430, described above.

In some embodiments, rotatable member 508 may be rotatable about a central axis C of an annular ring 520 (having any of the properties of annular ring 420). As with actuation mechanism 400, annular ring 520 may be disposed within handle 112 (FIGS. 1A-1B), and control lever 530 may extend outwardly through a housing of handle 112 via, e.g., a slit or opening.

Annular ring 520 may be rotatable relative to housing 113 of handle 112, such that movement of control lever 530 causes rotation of annular ring 520 relative to housing 13 of handle 112. Annular ring 520 and control lever 530 may be a single, unitary structure or may be separate components attached to one another.

Components of rotatable member 508 may be formed of any suitable material. For example, components of rotatable member 508 may be formed of rigid materials, such as plastic or other polymers, composites, or metal. Control lever 530, and annular ring 520 may be formed from the same materials or from different materials.

In some embodiments, a cover 528 may cover at least a portion of annular ring 520. Cover 528 may include all of the features of cover 428, described above.

A proximal end of plunger 504 may be coupled to rotatable member 508 at a connection 507. In some embodiments, a pin may secure plunger 504 to rotatable member 508 such that plunger 504 is rotatable relative to rotatable member 508. Plunger 504 may extend distally from rotatable member 408 within an interior of handle 112. In some embodiments, a distal end of plunger 504 may be coupled to a slider 542. A pin or other structure may rotatably couple the distal end of plunger 504 to slider 542 for example at connection point 560. Slider 542 may be coupled to a control member 546, having any of the properties of control member 446. Slider 542 may travel along a channel 544, having any of the properties of channel 444. Slider 542 and control member 546 may function in any manner described above for slider 442 and control member 446. Thus, plunger 504 may function in a manner similarly to linkage rod 440, causing an element coupled to a distal end of control member 446 to move or otherwise be actuated as rotatable member 508 rotates. Plunger 504 may be formed of any suitable material. For example, plunger 504 may be formed of rigid materials, such as plastic or other polymers, composites, or metal.

In some embodiments, a spring 512, for example a coil spring, may surround plunger 504 in a similar manner to how springs 412, 412′ surround plungers 404, 404′, respectively, described above. Thus, spring 512 may be indirectly coupled to rotatable member 508 via plunger 504. Spring 512 may similarly exert a force on a protrusion 509 (e.g., a force at least partially in the proximal direction), thereby exerting a torque on rotatable member 508 in configurations in which a longitudinal axis of plunger 504 does not pass through rotational axis C.

When an operator engages actuation mechanism 500 by rotating control lever 530, rotatable member 508 may rotate in response. Rotation of rotatable member 458 may cause a proximal end of plunger 504 to move in a circumferential or arcuate manner, as described above. The changing angle/position of plunger 504 may exert a torque on rotatable member 508, thereby lessening a torque required by an operator to move control lever 530. Movement of plunger 504 may simultaneously serve to move control member 546 proximally and/or distally. Thus, actuation mechanism 500 may, for example, raise or lower an elevator, such as elevator 126, or actuate an alternative element of a medical device.

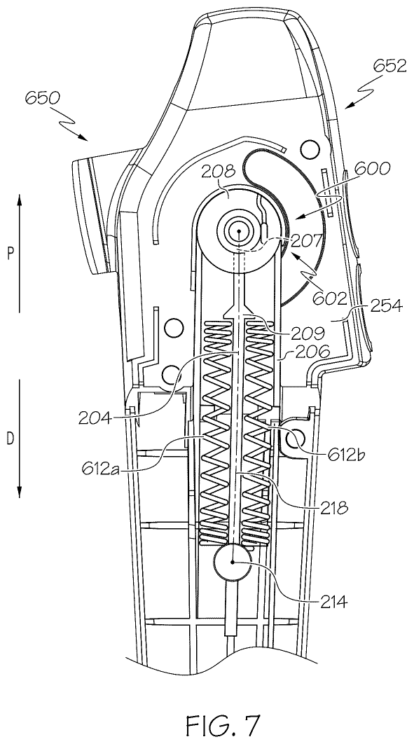

FIG. 7 shows an exemplary actuation assembly/mechanism 600, which may have any of the properties of actuation mechanism 200, unless otherwise specified herein. Although FIG. 7 depicts an actuation mechanism 600 that may be used to articulate a steerable section, such as steerable section 128, it will be appreciated that the features discussed herein are broadly applicable to actuators of medical devices that may be rotated in order to control one or more aspects of the medical device. Actuation mechanism 600 may be configured to decrease a torque experienced by an operator in operating actuation mechanism 600. As shown in FIG. 2, actuation mechanism may be at least partially disposed within housing 254 of a handle 652 of a medical device 650. Handle 652 may have any of the features of handle 252, except as specified herein. Medical device 650 may further include an insertion portion (not shown) extending distally from handle 652 and having any of the features of insertion portion 114, discussed above.

In some embodiments, for example as shown in FIG. 7, actuation mechanism 600 may include a pulley system 602, having any of the properties of pulley system 202, unless otherwise specified. Pulley system 602 may include rotatable member 208. In some embodiments, as shown in FIG. 7, at least one wire or cable 206 (or chain or belt) may be coupled to rotatable member 208. For example, as shown in FIG. 7, cable(s) 206 may wrap around rotatable member 208 (e.g., a proximal portion of rotatable member 208), and may be attached to rotatable member 208, as shown and described with respect to FIG. 2.

Rotatable member 208 also may be coupled to plunger 204, as described above, with respect to FIG. 2. A distal portion of plunger 204 may extend through or around an anchor 214, as discussed above. As also discussed above, a portion of plunger 204 (e.g., a proximal portion of plunger 204) may include protrusion 209.

Actuation mechanism 600 may include two springs 612a, 612b. In alternatives, actuation mechanism 600 may include additional springs. In some examples, springs 612a, 612b may be coiled springs, such as a helical compression spring. Springs 612a, 612b may extend alongside plunger 204. For example, springs 612a, 612b may extend on opposite sides of rod portion 218 of plunger 204 from one another. Longitudinal axes of springs 612a, 612b may be approximately parallel to a longitudinal axis of plunger 204. A proximal end of each of springs 612a, 612b may abut a distal surface of protrusion 209. A distal end of each of springs 612a, 612b may abut a proximal surface of anchor 214. In some examples, one or both ends of springs 612a, 612b may extend through openings or the like formed on plunger 204 in order to couple springs 612a, 612b to plunger 204. Thus, springs 612a, 612b may be indirectly coupled to rotatable member 208 via plunger 204.

Springs 612a, 612b may help to decrease a force required to rotate a control knob coupled to rotatable member 208, as discussed above for actuation mechanism 200. In some examples, using two springs 612a, 612b may increase a force exerted by springs 612a, 612b as compared with using one spring 212. In alternatives, springs 612a, 612b may have different properties (e.g., spring constants, lengths, etc.) than spring 212, such that, together, springs 612a, 612b exert a force similar to spring 212. In some examples, one of springs 612a, 612b may be omitted.

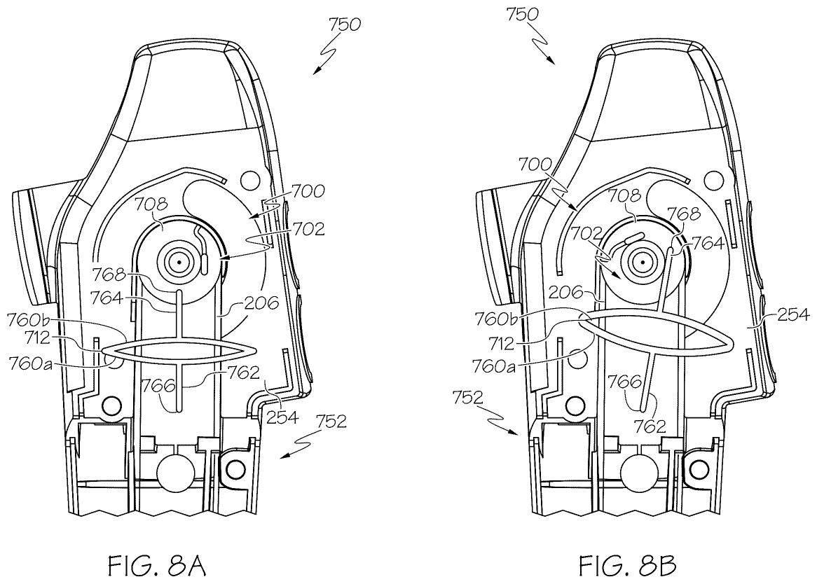

FIGS. 8A and 8B show an exemplary actuation assembly/mechanism 700, which may have any of the properties of actuation mechanism 200, unless otherwise specified herein. Although FIGS. 8A and 8B depict an actuation mechanism 700 that may be used to articulate a steerable section, such as steerable section 128, it will be appreciated that the features discussed herein are broadly applicable to actuators of medical devices that may be rotated in order to control one or more aspects of the medical device. Actuation mechanism 700 may be configured to decrease a torque experienced by an operator in operating actuation mechanism 700. As shown in FIGS. 8A and 8B, actuation mechanism may be at least partially disposed within housing 254 of a handle 752 of a medical device 750. Handle 752 may have any of the features of handle 252, except as specified herein. Medical device 750 may further include an insertion portion (not shown) extending distally from handle 752 and having any of the features of insertion portion 114, discussed above.

In some embodiments, for example as shown in FIGS. 8A and 8B, actuation mechanism 700 may include a pulley system 702, having any of the properties of pulley system 202, unless otherwise specified. Pulley system 702 may include rotatable member 708, having any of the properties of rotatable member 208, unless otherwise specified. In some embodiments, as shown in FIGS. 8A and 8B, at least one wire or cable 206 (or chain or belt) may be coupled to rotatable member 708. For example, as shown in FIGS. 8A and 8B, cable(s) 206 may wrap around rotatable member 708 (e.g., a proximal portion of rotatable member 708), as shown and described above with respect to rotatable member 208 of FIG. 2, and may be attached to rotatable member 708. As shown in FIGS. 8A and 8B, actuation mechanism 700 may not include a plunger, such as plunger 204, although such a feature may be present in some aspects.

Actuation mechanism 700 may include a spring 712. In some examples, spring 712 may be one or more leaf springs. For example, spring 712 may include a first leaf portion 760a and a second leaf portion 760b. First leaf portion 760a and second leaf portion 760b may be joined together at their ends. In alternatives, spring 712 may include only one leaf portion. A first leg 762 may extend (e.g., distally) from first leaf portion 760a. First leg 762 may be coupled to first leaf portion 760a at approximately a midpoint of first leaf portion 760a, although such an arrangement is only exemplary. A second leg 764 may extend (e.g., proximally) from second leaf portion 760b. Second leg 764 may be coupled to second leaf portion 760b at approximately a midpoint of first leaf portion 760b, although such an arrangement is only exemplary.

An end 766 of first leg 762 (which may be opposite to an end of first leg 762 that is coupled to first leaf portion 760a) may be coupled to housing 254. For example, end 766 may be pivotably coupled to housing 254 via opening(s) in housing 254, hinges, pins, or other structures. An end 768 of second leg 764 (which may be opposite to an end of second leg 764 that is coupled to second leaf portion 760b) may be pivotally coupled to rotatable member 708. For example, rotatable member 708 may include one or more openings to receive end 768 and/or one or more hinges, pins, or other structures may couple end 768 to rotatable member 708. End 768 may be coupled to rotatable member 708 at a location that has any of the properties of connection 205, discussed above. Thus, spring 712 may be directly or indirectly coupled to rotatable member 708.

FIG. 8A shows actuation mechanism 700 in an unactuated or neutral configuration (similar to the configuration of actuation mechanism 200 in FIG. 3A). In the neutral configuration, spring 712 may have a compressed configuration. In the configuration of FIG. 8A, a force exerted by spring 712 may be directed parallel to a lever arm of rotatable member 708. Thus, spring 712 may not exert a torque on rotatable member 708. Because the concepts of the force exerted by spring 712 are similar to the forces exerted by spring 212, discussed in detail above, such detail is not repeated for spring 712.

FIG. 8B shows actuation mechanism 700 in an actuated configuration, as rotatable member 708 has been rotated (e.g., via a knob, as discussed above for FIG. 2). In the configuration of FIG. 8B, spring 712 may exert a force that is not parallel to the lever arm of rotatable member 708. For example, spring 712 may exert a force substantially in a direction of a line that extends through legs 762 and 764 and/or a line that extends through midpoints of leaf portions 760a, 760b. This line may be a longitudinal axis of spring 712. As discussed above for actuation mechanism 200, among others, spring 712 may thus exert a torque on rotatable member 708, which may help to decrease an amount of force required by an operator to rotate rotatable member 708 (e.g., via an actuator, such as a knob or a lever).

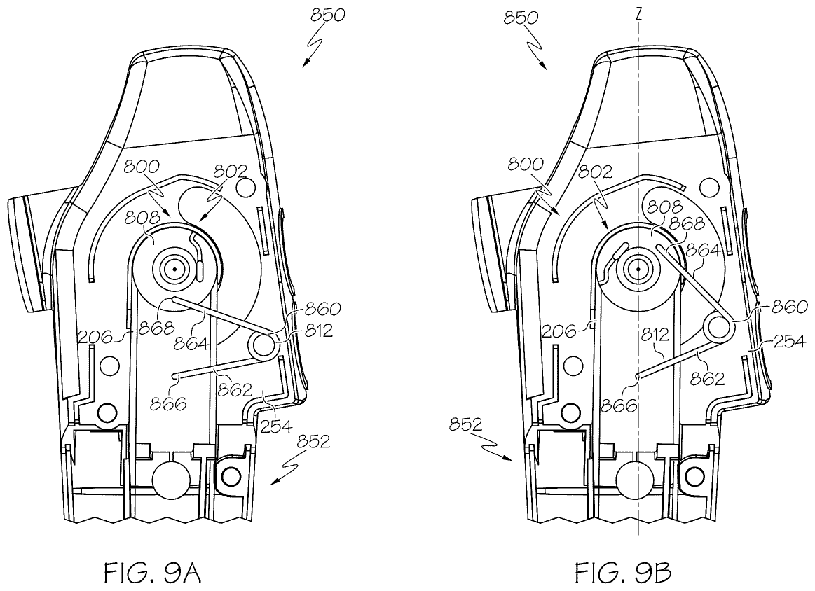

FIGS. 9A and 9B show an exemplary actuation assembly/mechanism 800, which may have any of the properties of actuation mechanisms 200, 700, unless otherwise specified herein. Although FIGS. 9A and 9B depict an actuation mechanism 800 that may be used to articulate a steerable section, such as steerable section 128, it will be appreciated that the features discussed herein are broadly applicable to actuators of medical devices that may be rotated in order to control one or more aspects of the medical device. Actuation mechanism 800 may be configured to decrease a torque experienced by an operator in operating actuation mechanism 800. As shown in FIGS. 9A and 9B, actuation mechanism may be at least partially disposed within housing 254 of a handle 852 of a medical device 850. Handle 852 may have any of the features of handles 252, 752, except as specified herein. Medical device 850 may further include an insertion portion (not shown) extending distally from handle 852 and having any of the features of insertion portion 114, discussed above.

In some embodiments, for example as shown in FIGS. 9A and 9B, actuation mechanism 800 may include a pulley system 802, having any of the properties of pulley systems 202, 702, unless otherwise specified. Pulley system 802 may include rotatable member 808, having any of the properties of rotatable member 208, 708, unless otherwise specified. In some embodiments, as shown in FIGS. 9A and 9B, at least one wire or cable 206 (or chain or belt) may be coupled to rotatable member 808, as shown and described above for rotatable member 208 and cable 206 in FIG. 2. For example, as shown in FIGS. 9A and 9B, cable(s) 206 may wrap around rotatable member 808 (e.g., a proximal portion of rotatable member 808), and may be attached to rotatable member 808. As shown in FIGS. 9A and 9B, actuation mechanism 800 may not include a plunger, such as plunger 204, although such a feature may be present in some aspects.

Actuation mechanism 800 may include a spring 812. In some examples, spring 812 may be one or more hinge springs. For example, spring 812 may include a central portion 860, which may include one or more loops. A first leg 862 of spring 812 may extend (e.g., at least partially distally) from central portion 860. A second leg 864 of spring 812 may extend (e.g., at least partially proximally) from central portion 860. Central portion 860 may be positioned off-center relative to a central longitudinal axis Z (FIG. 9B) of handle 850 that extends through a rotational axis of rotatable member 808.

An end 866 of first leg 862 (which may be opposite to an end of first leg 862 that is coupled to central portion 860) may be coupled to housing 254. For example, end 866 may be pivotably (or, in some examples, fixedly) coupled to housing 254 via opening(s) in housing 254, hinges, pins, or other structures. In examples, end 866 may be coupled to housing 254 at or near central longitudinal axis Z. An end 868 of second leg 864 (which may be opposite to an end of second leg 864 that is coupled to central portion 860) may be pivotally coupled to rotatable member 808. For example, rotatable member 808 may include one or more openings to receive end 868 and/or one or more hinges, pins, or other structures may couple end 868 to rotatable member 808. End 868 may be coupled to rotatable member 808 at a location that has any of the properties of connection 205, discussed above. Thus, spring 812 may be directly or indirectly coupled to rotatable member 808.

FIG. 9A shows actuation mechanism 800 in an unactuated or neutral configuration (similar to the configuration of actuation mechanism 200 in FIG. 3A). In the neutral configuration, spring 812 may have a compressed configuration. In the configuration of FIG. 8A, a force exerted by spring 812 may be directed parallel to a lever arm of rotatable member 808. For example, a direction of a force exerted by spring may extend along a line that extends between ends 866 and 888. Thus, spring 812 may not exert a torque on rotatable member 808. Because the concepts of the force exerted by spring 812 are similar to the forces exerted by spring 212, discussed in detail above, such detail is not repeated for spring 812.

FIG. 9B shows actuation mechanism 800 in an actuated configuration. For example, rotatable member 808 may be rotated via, e.g., a knob or other actuator, as described above. In the configuration of FIG. 9B, spring 812 may exert a force that is not parallel to the lever arm of rotatable member 808. For example, spring 812 may exert a force substantially in a direction of a line that extends through ends 866 and 888. This line may be a longitudinal axis of spring 812. As discussed above for actuation mechanism 200, among others, spring 812 may thus exert a torque on rotatable member 808, which may help to decrease an amount of force required by an operator to rotate rotatable member 808 (e.g., via an actuator, such as a knob or a lever).

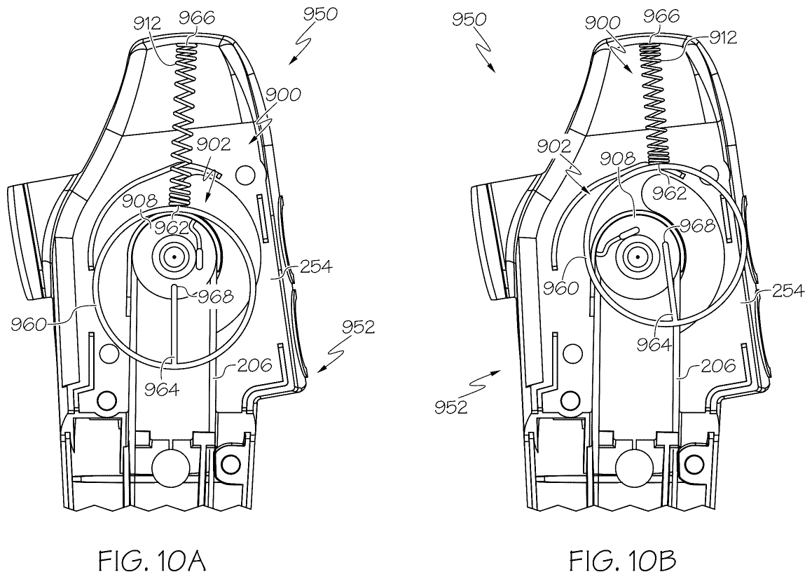

FIGS. 10A and 10B show an exemplary actuation assembly/mechanism 900, which may have any of the properties of actuation mechanisms 200, 700, 800, unless otherwise specified herein. Although FIGS. 10A and 10B depict an actuation mechanism 900 that may be used to articulate a steerable section, such as steerable section 128, it will be appreciated that the features discussed herein are broadly applicable to actuators of medical devices that may be rotated in order to control one or more aspects of the medical device. Actuation mechanism 900 may be configured to decrease a torque experienced by an operator in operating actuation mechanism 900. As shown in FIGS. 10A and 10B, actuation mechanism may be at least partially disposed within housing 254 of a handle 952 of a medical device 950. Handle 952 may have any of the features of handles 252, 752, 852, except as specified herein. Medical device 950 may further include an insertion portion (not shown) extending distally from handle 952 and having any of the features of insertion portion 114, discussed above.

In some embodiments, for example as shown in FIGS. 10A and 10B, actuation mechanism 900 may include a pulley system 902, having any of the properties of pulley systems 202, 702, 802, unless otherwise specified. Pulley system 902 may include rotatable member 908, having any of the properties of rotatable member 208, 708, 808, unless otherwise specified. In some embodiments, as shown in FIGS. 10A and 10B, at least one wire or cable 206 (or chain or belt) may be coupled to rotatable member 908, as shown and described above for rotatable member 208 and cable 206 (FIG. 2). For example, as shown in FIGS. 9A and 9B, cable(s) 206 may wrap around rotatable member 908 (e.g., a proximal portion of rotatable member 808), and may be attached to rotatable member 908. As shown in FIGS. 10A and 10B, actuation mechanism 900 may not include a plunger, such as plunger 204, although such a feature may be present in some aspects.

Actuation mechanism 900 may include a spring 912. In some examples, spring 912 may be a coil spring having any of the properties of spring 212. Actuation mechanism 900 may also include a ring 960. Although a circular ring 960 is depicted, ring 960 may include only a portion of a circumference of a circle or may have a non-circular shape or another arcuate shape. A first end 962 (e.g., a distal end) of spring 912 may be pivotably (or, in some examples, fixedly) coupled to ring 960. A second end 966 (e.g., a proximal end) of spring 912 may be pivotably (or, in some examples, fixedly) coupled to housing 254 in any suitable manner.

In some examples, spring 912 may be proximal of rotatable member 908, and ring 960 may be distal of spring 912, such that ring 960 at least partially surrounds rotatable member 908 in at least some configurations of actuation mechanism 900. A leg 964 may extend from ring 960 toward a center of ring 960 (i.e., radially inwardly within ring 960). Leg 964 may extend a long a portion of a diameter of ring 960. For example, leg 964 may extend along an entire radius of ring 960. Leg 964 may be coupled to ring 960 at a point that is approximately diametrically opposed to a point where firs tend 962 of spring 912 is coupled to ring 960. However, such an arrangement is exemplary, and other arrangements are possible.

An end 968 of leg 964 that is opposite to an end of leg 964 that is coupled to ring 960 may be pivotably coupled to rotatable member 908. End 968 may be within a circle defined by ring 960 and, in some examples, may be at or near a center point of a circle defined by ring 960. For example, rotatable member 908 may include one or more openings to receive end 968 and/or one or more hinges, pins, or other structures may couple end 968 to rotatable member 908. End 968 may be coupled to rotatable member 908 at a location that has any of the properties of connection 205, discussed above. Thus, spring 912 may be indirectly coupled to rotatable member 808 via ring 960 and leg 964.

FIG. 10A shows actuation mechanism 900 in an unactuated or neutral configuration (similar to the configuration of actuation mechanism 200 in FIG. 3A). In the neutral configuration, spring 912 may have an elongated configuration, as compared to a neutral length of spring 912. In the configuration of FIG. 9A, a force exerted by spring 912 may be directed parallel to a lever arm of rotatable member 908. For example, a direction of a force exerted by spring may extend along a longitudinal axis of spring 912 and/or leg 964. Thus, spring 912 may not exert a torque on rotatable member 908. Because the concepts of the force exerted by spring 912 are similar to the forces exerted by spring 212, discussed in detail above, such detail is not repeated for spring 912.

FIG. 10B shows actuation mechanism 900 in an actuated configuration. For example, rotatable member 908 may have been rotated by ay of the mechanisms described above. In the configuration of FIG. 10B, spring 912 may exert a force that is not parallel to the lever arm of rotatable member 908. For example, spring 912 may exert a force substantially in a direction of a longitudinal axis of spring 912 (and, in some examples, arm 964). As discussed above for actuation mechanism 200, among others, spring 912 may thus exert a torque on rotatable member 908, which may help to decrease an amount of force required by an operator to rotate rotatable member 908 (e.g., via an actuator, such as a knob or a lever).

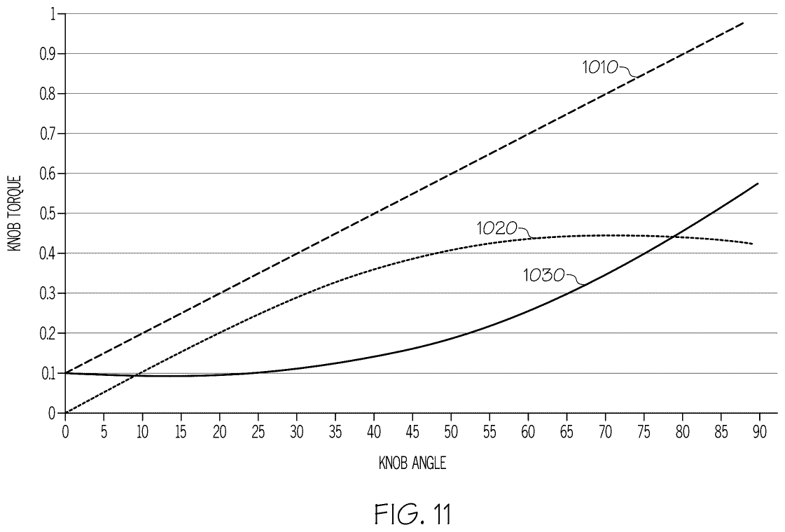

FIG. 11 shows a graph depicting aspects of the disclosed assemblies according to some embodiments. Specifically, the plot shown in FIG. 11 depicts the torque perceived by an operator of a duodenoscope as a function of the knob angle, for example control knobs 132/134, elevator control mechanism 138, or control levers 430/530, which may be coupled to any of actuation mechanisms described herein. Line 1010 (which may alternatively be a curve) represents the typical knob torque in a typical device (i.e., a device not utilizing an actuation mechanism such as those described herein). Curve 1020 depicts a torque exerted by a spring of the actuation mechanisms disclosed herein (e.g., spring 212, 212′, 212″, 412, 412′, 512, 612a/612b, 712, 812, or 912). As shown in FIG. 11, as a knob angle increases, a torque exerted by the spring increases, as discussed above (e.g., with respect to FIGS. 3A-3C). Curve 1030 depicts a magnitude of a torque perceived by an operator using the knob. A direction of torque generated by cables, linkages, etc. coupled to an actuation mechanism may be opposite to a direction generated by any of the springs above. It will be appreciated that rotation in an opposite direction may result in a torque with an opposite direction (and may have a negative value in some examples). Curve 1030 may be equal to line 1010 minus curve 1020. As shown, although the perceived torque does increase as the knob angle increases along curve 1020, it does not increase nearly as rapidly or as drastically as the torque line 1010. Accordingly, the spring (e.g., spring 212, 212′, 212″, 412, 412′, 512, 612a/612b, 712, 812, or 912) acts to significantly reduce the perceived torque to help reduce user fatigue.

While principles of this disclosure are described herein with reference to illustrative examples for particular applications, it should be understood that the disclosure is not limited thereto. For example, the disclosure refers to duodenoscopes as an exemplary scope including a handle assembly. The systems, devices, and methods of the present disclosure, however, may be used in any suitable scope device. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. An actuation mechanism for a medical device, the actuation mechanism comprising:
a rotatable member configured to rotate about a rotational axis; and
a spring, coupled directly or indirectly to the rotatable member,
wherein the spring is configured to move from a first configuration to a second configuration, wherein, in the first configuration, a longitudinal axis of the spring has a first angle with respect to a lever arm of the rotatable member, and wherein, in the second configuration, a longitudinal axis of the spring has a second angle with respect to the lever arm of the rotatable member.

2. The actuation mechanism of claim 1, further comprising a plunger coupled to the rotatable member at a connection, wherein a portion of the plunger extends through a lumen of the spring.

3. The actuation mechanism of claim 2, further comprising an anchor, wherein a distal end of the spring is coupled to the anchor, and wherein the plunger extends through and is movable relative to the anchor.

4. The actuation mechanism of claim 2, wherein the plunger includes a protrusion, and wherein a proximal end of the spring engages with the protrusion.

5. The actuation mechanism of claim 1, wherein the first angle is smaller than the second angle, and wherein a first torque exerted by the spring on the rotatable member in the first configuration is smaller than a second torque exerted by the spring on the rotatable member in the second configuration.

6. The actuation mechanism of claim 1, wherein, in a relaxed configuration of the actuation mechanism, a longitudinal axis of the spring is approximately coaxial with the lever arm of the rotatable member.

7. The actuation mechanism of claim 1, wherein the rotatable member includes a pulley, and wherein the actuation mechanism further includes a wire, cable, chain, or belt coupled to the pulley.

8. The actuation mechanism of claim 1, wherein the actuation mechanism further includes a linkage arm coupled to the rotatable member, wherein the linkage arm is coupled to a control wire.

9. The actuation mechanism of claim 8, further comprising a plunger coupled to the rotatable member at a connection, wherein a portion of the plunger extends through a lumen of the spring, and wherein the linkage arm is coupled to the rotatable member at the connection.

10. The actuation mechanism of claim 9, wherein the connection is a first connection, and wherein the linkage arm is coupled to the rotatable member at a second connection, wherein the second connection is at a different location than the first connection.

11. The actuation mechanism of claim 9, wherein a distal end of the plunger is coupled to a control wire.

12. The actuation mechanism of claim 1, wherein the spring is a coil spring, a leaf spring, or a hinge spring.

13. The actuation mechanism of claim 1, wherein the rotatable member is a first rotatable member, wherein the lever arm is a first lever arm, and wherein the spring is a first spring, the actuation mechanism further comprising:
a second rotatable member configured to rotate about the rotational axis; and
a second spring directly or indirectly to the rotatable member,
wherein the second spring is configured to move from a first configuration to a second configuration, wherein, in the first configuration, a longitudinal axis of the second spring has a first angle with respect to a second lever arm of the second rotatable member, and wherein, in the second configuration, a longitudinal axis of the second spring has a second angle with respect to the second lever arm of the second rotatable member.

14. The actuation mechanism of claim 1, wherein the rotatable member is coupled to a lever or a knob.

15. The actuation mechanism of claim 1, wherein a torque exerted by the spring on the rotatable member in the second configuration decreases an amount of force required for an operator to actuate the rotatable member.

16. An actuation mechanism for a medical device, the actuation mechanism comprising:
a rotatable member configured to rotate about a rotational axis; and
a spring configured to exert a force on the rotatable member;
wherein the spring is configured to move from a first configuration to a second configuration; wherein, in the first configuration, the spring exerts a force along a first force vector, such that a first lever arm between the first force vector and the rotational axis has a first length; wherein, in the second configuration, the spring exerts a force along a second force vector, such that a second lever arm between the second force vector and the rotational axis has a second length, and wherein the first length is smaller than the second length.

17. The actuation mechanism of claim 16, further comprising a plunger, wherein the plunger is coupled to the rotatable member, and wherein the plunger extends through a lumen of the spring.

18. The actuation mechanism of claim 17, wherein a distal end of the plunger extends through an anchor, and wherein a distal end of the spring is coupled to the anchor.

19. An actuation mechanism for a medical device, the actuation mechanism comprising:
a rotatable member configured to rotate about a rotational axis;
a plunger coupled to the rotatable member at a connection;
a spring, wherein a portion of the plunger extends through a lumen of the spring; and
an anchor coupled to a distal end of the spring;
wherein, as the rotatable member rotates about the rotational axis, the connection follows an arcuate or circular path around the rotational axis.

20. The actuation mechanism of claim 19, wherein, as the rotatable member rotates about the rotational axis, a distance between a force vector of the spring and the rotational axis changes.
